(19) United States
(12) Patent Application Publication
(10) Pub. No.: US 2024/0355425 A1
(43) Pub. Date: Oct. 24, 2024

(54) METHOD AND SYSTEM FOR IDENTIFYING AND UTILIZING FRUGAL MARKERS FOR CLASSIFICATION OF BIOLOGICAL SAMPLE

(71) Applicant: Tata Consultancy Services Limited, Mumbai (IN)

(72) Inventors: MOHAMMED MONZOORUL HAQUE, Pune (IN); RASHMI SINGH, Pune (IN); MITALI MERCHANT, Pune (IN); SHARMILA SHEKHAR MANDE, Pune (IN); VENKATA SIVA KUMAR REDDY CHENNAREDDY, Pune (IN); SUNIL NAGPAL, Delhi (IN); ANIRBAN DUTTA, Pune (IN)

(73) Assignee: Tata Consultancy Services Limited, Mumbai (IN)

(21) Appl. No.: 18/426,000

(22) Filed: Jan. 29, 2024

(30) Foreign Application Priority Data
Apr. 19, 2023 (IN) 202321028613

(51) Int. Cl. G16B 40/20; G06N 20/20

(52) U.S. Cl. CPC G16B 40/20; G06N 20/20

(57) ABSTRACT

The present disclosure is related to method and system for identifying and utilizing frugal markers for classification of biological sample. Discovering an optimal and/or frugal set of features/biomarkers form a large set of features measured through high-throughput screening techniques, which can characterize a disease/anomaly with sufficient accuracy, still remains a challenge. According to the present disclosure, given a set of measurements of multiple features characterizing biological samples obtained from disease cases and healthy controls, a classification model combining the measured values of a small subset of the features is computed. The classification model is then used for classifying between disease cases and healthy controls.

### PRIORITY CLAIM

This U.S. patent application claims priority under 35 U.S.C. § 119 to: Indian patent application No. 202321028613, filed on Apr. 19, 2023. The entire contents of the aforementioned application are incorporated herein by reference.

### TECHNICAL FIELD

The disclosure herein generally relates to the field of a biomarker identification and, more particularly, to a method and system for identifying and utilizing frugal markers for classification of biological sample.

### BACKGROUND

Search for novel biomarkers to diagnose the occurrence or progression of non-communicable disease or assessing therapeutic response (patient stratification), is an active field of research. Technological advances in the fields of microfluidics and imaging, applied to the domain of biochemistry and life sciences, has enabled high throughput screening of multiple biochemicals, metabolites, gene-expressions, oligonucleotides etc. from a biological/environmental sample, using fewer experiments. These advances have therefore driven research towards search for diagnostic solutions that can rely on multiple biomarkers for a disease diagnosis, given that multiple biomarkers in combination are expected to provide more accurate results than diagnostic solutions relying on single biomarkers.

Many next-generation IVD (in-vitro diagnostic) solutions employing this strategy have enabled assessment of diseases/disorders which would otherwise remain undetected. However, discovering an optimal and/or frugal set of features/biomarkers form a large set of features measured through high-throughput screening techniques, which can characterize a disease/anomaly with sufficient accuracy, remains a challenge.

### SUMMARY

Embodiments of the present disclosure present technological improvements as solutions to one or more of the above-mentioned technical problems recognized by the inventors in conventional systems.

In one aspect, a method for identifying and utilizing a frugal set of markers for classification of a test biological sample is provided. The method comprising: collecting a plurality of train biological samples comprising of a first subset of samples corresponding to a first class and a second subset of samples corresponding to a second class; extracting a microbial Deoxyribonucleic Acid (DNA) from each of the plurality of train biological samples; sequencing the microbial DNA associated with each of the plurality of train biological samples, using a sequencer, to obtain a DNA sequence data corresponding to each of the plurality of train biological samples; analyzing the DNA sequence data corresponding to each of the plurality of train biological samples, to generate a plurality of microbial abundance profiles from the plurality of train biological samples, wherein each of the plurality of microbial abundance profiles corresponds to each of the plurality of train biological samples and comprises of abundance values associated with a plurality of individual microbial taxonomic groups corresponding to each of the plurality of train biological samples; building, an ensemble classification model by training a machine learning (ML) model using the plurality of microbial abundance profiles associated with the plurality of train biological samples, wherein the ensemble classification model comprises of a set of no more than a predefined number of microbial taxonomic groups as the frugal set of markers, wherein the set of no more than the predefined number of microbial taxonomic groups is a subset of the plurality of individual microbial taxonomic groups, and wherein the ensemble classification model comprises of one or more classification sub-models and is configured to classify the test biological sample to one of the first class and the second class; designing a set of quantitative polymerase chain reaction (qPCR)-probes corresponding to each microbial taxonomic group constituting to the frugal set of markers; determining, a minimum number of multiplexed qPCR runs required for quantifying a relative abundance of each microbial taxonomic group belonging to the frugal set of markers, based on a unique number of microbial taxonomic groups constituting the frugal set of markers, wherein each multiplexed qPCR run is configured to determine the relative abundance of the predetermined subset of the microbial taxonomic groups constituting the frugal set of markers, in the test biological sample; determining, a ranking of each of the microbial taxonomic groups constituting the frugal set of markers, based on one or more of (i) a median abundance of each of the frugal set of markers from the plurality of train biological samples, and (ii) a frequency of occurrence of each of the frugal set of markers across the one or more classification sub-models constituting the ensemble classification model, wherein the ranking is utilized for determining the subset of microbial taxonomic groups from amongst the taxonomic groups constituting the frugal set of markers whose abundance is to be probed, in the test biological sample, using more than one multiplexed qPCR runs; determining the relative abundance of each of the microbial taxonomic groups constituting the frugal set of markers in the test biological sample, by performing the determined minimum number of multiplexed qPCR runs utilizing a designed set of qPCR probes, and the ranking of each of the microbial taxonomic groups constituting the frugal set of markers; and classifying, the test biological sample to one of the first class and the second class, utilizing the ensemble classification model, based on the relative abundance of each of the microbial taxonomic groups constituting the frugal set of markers in the test biological sample (220).

In an embodiment, the minimum number of multiplexed qPCR runs is determined using a formula: 1+┌(n−4)/4┐, where n is the unique number of microbial taxonomic groups constituting the frugal set of markers.

In an embodiment, the ensemble classification model is built by:
(i) assigning one of a first class tag or a second class tag to each of the plurality of training biological samples;
(ii) generating a training data comprising a plurality of microbial abundance profiles from the plurality of training biological samples, wherein each microbial abundance profile corresponds to each of the plurality of training biological samples and comprises of one or more features and respective abundance values, and wherein each feature in the associated microbial abundance profile corresponds to one of a plurality of microbial taxonomic groups present in the associated training biological sample;
(iii) partitioning the training data into an internal training set and an internal test set, based on a predefined first parameter;
(iv) randomly selecting a predefined number of subsets out of the internal training set based on a predefined second parameter, wherein each subset comprises of a randomly selected plurality of microbial abundance profiles corresponding to the training biological samples in the randomly selected subset, and wherein each subset comprises of a proportionate part of training biological samples belonging to the first class and the remaining training biological samples belonging to the second class;
(v) noting, for each selected subset, a distribution of the abundance values of each of the features across the plurality of training biological samples in the selected subset, and the distribution of the abundance values of each of the features across the training biological samples belonging to the first class in the selected subset and the training biological samples belonging to the second class in the selected subset;
(vi) calculating, from the noted distributions of each selected subset, a first quartile value Q1 and a third quartile value Q3 of the distribution of each of the features across each of the plurality of training biological samples in the selected subset;
(vii) calculating, for each selected subset, a second quartile value of the distribution of each of the features across the training biological samples belonging to the first class Q2_{A} in the selected subset and the training biological samples belonging to the second class Q2_{B} in the selected subset;
(iii) calculating Q1, Q3, Q2_{A} and Q2_{B} for each of a predefined number of subsets M;
(ix) calculating a median value for each of the Q1, Q3, Q2_{A} and Q2_{B};
(x) performing a Mann-Whitney test to check whether the median value (Q2_{A}) of the feature in the training biological samples belonging to the first class is significantly different as compared to the median value (Q2_{B}) of the associated feature in the training biological samples belonging to the second class;
(xi) shortlisting the features based on a first predefined criteria utilizing calculated median values and the Mann-Whitney test;
(xii) generating a set of features using the shortlisted features using a second predefined criteria, wherein the set of features are less than or equal to a predefined second criteria value;
(xiii) creating a plurality of combinations of the features present in the set of features to generate a plurality of candidate feature sets, wherein a number of the plurality of combinations of the features is equal to a minimum of two and a maximum of the predefined second criteria value;
(xiv) building a plurality of candidate models (CM_{K}) corresponding to each of the plurality of candidate feature sets; (xv) calculating a model evaluation score (MES) corresponding to each of the plurality of candidate models;
(xvi) selecting a model having a highest MES, out of the plurality of candidate models as a best model, based on a first threshold (Tₘₐₓ), wherein the selected model is tagged as a forward model; (xvii) swapping the first class tag and the second class tag assigned to the first class and the second class of the plurality of training biological samples present in the training data;
(xviii) identifying and subsequently tagging the model as a reverse model by repeating the steps (ii) through (xvi) for the training data obtained after swapping the first class tag and the second class tag;
(xix) generating a plurality of forward models and a plurality of reverse models by repeating step (ii) through (xviii) for a predefined number of times using randomly created partitions of internal training sets and corresponding internal test sets from the training data;
(xx) generating an ensemble of forward models (ENS-MD_{fwd}) using the plurality of forward models and an ensemble of reverse models (ENS-MDᵣₑᵥ) using the plurality of reverse models;
(xxi) identifying a best forward model and a best reverse model using the model evaluation score; and
(xxii) choosing a final single model (FM_{single}) from amongst the best forward models and the best reverse model, and a final ensemble classification model (FMₑₙₛ) from among the ensemble of forward models and the ensemble of reverse models, based on the classification of the individual training biological samples from the training data.

In an embodiment, building the ensemble classification model further comprises classifying each of the set of shortlisted features using a second threshold value different from the first threshold (Tₘₐₓ); and cumulating the results to construct a receiver operating characteristic curve (ROC) for each of the shortlisted features, wherein an area under the curve (AUC) of the ROC is indicative of utility of the feature to distinguish between the training biological samples belonging to the first class and the second class.

In an embodiment, calculating the model evaluation score (MES) comprises:
transforming the values of the set of features as follows:

$${F}_{j}^{′} = 0 … if {F}_{j} < {j}_{} \\ {F}_{j}^{′} = 1 … if {F}_{j} > {j}_{} \\ {F}_{j}^{′} = 0.5 … if {j}_{} = {j}_{} \\ {F}_{j}^{′} = … if {j}_{} < {F}_{j} < {j}_{} ;$$

collating the features out of the set of features as a set of numerator features (Fₙᵤₘₑᵣₐₜₒᵣ) if [symbol] > [symbol] , else, collating the features out of the set of features as a set of denominator features (F_{denominator});
constituting a ratio function for each of the candidate model as:

$${CM}_{K} = \frac{\sum {F}_{numerator}}{\sum {F}_{denominator}} … when {F}_{numerator} > 0 and {F}_{denominator} > 0 or , \\ {CM}_{K} = \frac{\sum {F}_{numerator} + 1}{\sum {F}_{denominator} + 1} … when either {F}_{numerator} or {F}_{denominator} = 0$$

wherein, ΣFₙᵤₘₑᵣₐₜₒᵣ represents the sum of values of all numerator features for a particular training biological sample, and,
wherein, ΣF_{denominator} represents the sum of values of all denominator features for a particular training biological sample;
   generating a candidate model score (CMS_{K}) for each of the training biological samples in the internal train set;
   removing the top 10 percentile and bottom 10 percentile scores as outliers from the set of scores CMS_{K}, and identifying maximum and minimum scores from the set CMS_{K} as CMS_{K}_{ₘₐₓ} and CMS_{K}_{ₘᵢₙ} respectively;
   reclassifying the training biological samples in the internal train set by considering each score in the set CMS as threshold, such that the training biological sample is classified into the second class B if CMS_{K} is more than or equal to the threshold, or the training biological sample is classified into the first class if CMS_{K} is less than the threshold;
   calculating Matthew's correlation coefficients (MCC) for each of the thresholds based on a comparison of the reclassified training biological sample and the original classes of the training biological samples, to evaluate how well each of the thresholds are able to distinguish between the training biological samples associated to the first class and the second class;
   identifying the threshold as a first threshold (Tₘₐₓ) which provides maximum absolute MCC value;
   discarding the candidate models for further evaluation if the maximum absolute MCC value is less than 0.4;
   considering the (|MCCₘₐₓ|) value as the ‘train-MCC’ value (MCCₜᵣₐᵢₙ) for the model CM_{K} and the model and the first threshold (Tₘₐₓ) is used to classify the training biological samples in the internal-test set;
   comparing the classification results on the training biological samples from the internal test set against the original classes of the training biological samples with pre-assigned labels, and the MCC for the model CM_{K} and the threshold Tₘₐₓ threshold on the internal train set is calculated (MCCₜₑₛₜ); and
   calculating a model evaluation score (MES) for candidate model

$${CM}_{K} as : MES = \vert ( {MCC}_{train} + {MCC}_{test} ) \vert - \vert ( {MCC}_{train} - {MCC}_{test} ) \vert .$$

In an embodiment, building the ensemble classification model further comprises evaluating collective classification efficiencies of the ensemble of forward models (ENS-MD_{fwd}) and the ensemble of reverse models (ENS-MDᵣₑᵥ), using an ensemble model scoring method, wherein a model scores (MS) corresponding to each of the ensemble is transformed into a scaled model scores (SMS) having values between −1 and +1, wherein,

$$SMS = ( MS - {T}_{max} ) / ( {CMS}_{{K}_{max}} - {T}_{max} ) , … when MS >= {T}_{max} , and \\ SMS = ( MS - {T}_{max} ) / ( {T}_{max} - {CMS}_{{K}_{min}} ) , … when MS < {T}_{max} ,$$

wherein, Tₘₐₓ, CMS_{K}_{ₘₐₓ} and CMS_{K}, values corresponding to the respective model.

In an embodiment, building the ensemble classification model further comprises calculating an average of all SMS (SMS_{avg}) obtained using all models in the ensemble, wherein
SMS_{avg}=SMS_{avg}*(+1) while using the ensemble of forward models (ENS-MD_{fwd}).
   If SMS_{avg}>=0, training biological sample is classified as the second class; and
   If SMS_{avg}<0, training biological sample is classified as the first class; and
SMS_{avg}=SMS_{avg}*(−1) while using the ensemble of reverse model (ENS-MDᵣₑᵥ),
   If SMS_{avg}>0, training biological sample is classified as the second class; and
   If SMS_{avg}<=0, training biological sample is classified as the first class.

In an embodiment, building the ensemble classification model further comprises selecting a final ensemble model (FMₑₙₛ) using the calculated SMS_{avg}, wherein the classification model is one of: the final single model (FM_{single}) or an ensemble of more than one classification models (FMₑₙₛ).

In an embodiment, the relative abundance of each of the microbial taxonomic groups constituting the frugal set of markers, that are common to each of the minimum number of the multiplexed qPCR runs, is determined based on a normalizing factor associated with each multiplexed qPCR run and the relative abundance of associated microbial taxonomic group in the corresponding multiplexed qPCR run.

In an embodiment, determining the relative abundance of each of the microbial taxonomic groups constituting the frugal set of markers in the test biological sample, comprises multiplying a ratio of inferred DNA concentrations of each marker of the frugal set of markers and an inferred DNA concentration of an anchor marker with the median abundance of the anchor marker across the plurality of training biological samples, wherein the anchor marker is selected from the frugal set of markers having a lowest variance in the associated relative abundance.

In an embodiment, each train biological sample and the test biological sample are selected from a group comprising of a vaginal swab sample, a cervical mucus sample, a cervical swab sample, a vaginal swab including swab sample of a vaginal fornix, a urine sample, an amniotic fluid sample, a blood sample, a serum sample, a plasma sample, a placental swab, an umbilical swab, a stool sample, a skin swab, an oral swab, a saliva sample, a periodontal swab, a throat swab, a nasal swab, a vesicle fluid sample, a nasopharyngeal swab, a nares swab, a conjunctival swab, a genital swab, a rectum swab, a tracheal aspirate, and a bronchial swab.

In an embodiment, one or more frugal markers from amongst the frugal set of markers that have a relatively higher median abundance or frequency of occurrence as compared to the median abundances or the frequency of occurrence of each frugal marker in the remaining frugal set of markers, across the plurality of training biological samples are common to the multiplex qPCR runs.

In an embodiment, the first predefined criteria is if a feature (Fⱼ) is observed to have significantly (p<0.1) different median values in the first class compared to the second class in >70% of predefined number of subsets, and if [symbol] >=Q2ₘᵢₙ or [symbol] >=Q2ₘᵢₙ, Fⱼ is added to a set of shortlisted features (SF).

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates a network diagram of a system for identifying and utilizing a frugal set of markers for classification of a test biological sample, according to some embodiments of the present disclosure.

FIGS. 2A and 2B are flowcharts illustrating a method for identifying and utilizing a frugal set of markers for classification of a test biological sample, according to some embodiments of the present disclosure.

FIGS. 3A, 3B, 3C are flowcharts illustrating the steps involved in building an ensemble classification model, according to some embodiments of the present disclosure.

FIG. 4 illustrates an exemplary multiplex qPCR design for detecting and quantifying microbial DNA having an exemplary set of qPCR-probes, according to some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

The existing methodologies for biomarker identification rely on different statistical methods as well as machine learning approaches, often ends up with classification models which are highly accurate but dependent on a significant higher number of features. On the other hand, classification models obtained using lesser number of features are often not accurate enough for clinical deployment. Although high-throughput screening methods can be used in biomarker discovery (and hypothesis testing) phases for screening/measuring a large number of features (and making observational inferences from measurements corresponding to the same), higher costs prevent deployment of the same for diagnostics at a population scale. In effect, the discovered (set of) biomarker(s) find clinical relevance only with sufficient accuracy of prediction and can be measured economically. Ideally, a diagnostic method should rely on a small number of biomarkers/features, which can be measured economically, and still provide sufficient accuracy.

Similarly, from the therapeutic perspective, biomarkers discovered assumes importance in cases where causal relationship of the biomarkers with the disease in question can be identified. Corrective measures to modulate the levels of the biomarkers can then be considered for effective disease management. Even in this scenario, identification of a smaller number of features (biomarkers) aid in practical therapeutic design.

According to an embodiment of the disclosure, a method provides a technical solution to the above-mentioned technical problems by identifying and utilizing a frugal set of markers for classification of a test biological sample.

The present disclosure provides a method for identification of a frugal combination of disease biomarkers that can enable accurate and easy assessment of a disease and guide in therapy. To be specific, given a set of measurements of multiple (physical/biochemical/genetic/microbiological etc.) features characterizing biological samples obtained from cohorts (a group of individuals) pertaining to disease cases and (healthy) controls, the present disclosure computes a metric (akin to a mathematical model) combining the measured values of a small subset (not exceeding 15) of the features (predetermined or preidentified), which can be used for classifying between case and control samples. Any newly obtained sample can be classified into the case or control group by using the defined metric, the computation of which only involves obtaining measured features corresponding to the pre-defined small subset (predetermined or preidentified). In effect, the method allows accurate diagnosis while using lesser (or frugal) number of features (implying lower cost of measurement), compared to methods taught by current state-of-art. By virtue of selecting a small set of features/descriptors which are characteristic of the disease, the method further enables an easy trace-back towards causality and guides in recommending or designing a focused and personalized therapeutic design. Relying on the “guilt by association” principle, the therapeutic design may involve correcting the anomalous abundance patterns of the identified subset of features either by directly spiking or antagonizing them or by modulating the abundance levels of their close correlates (features).

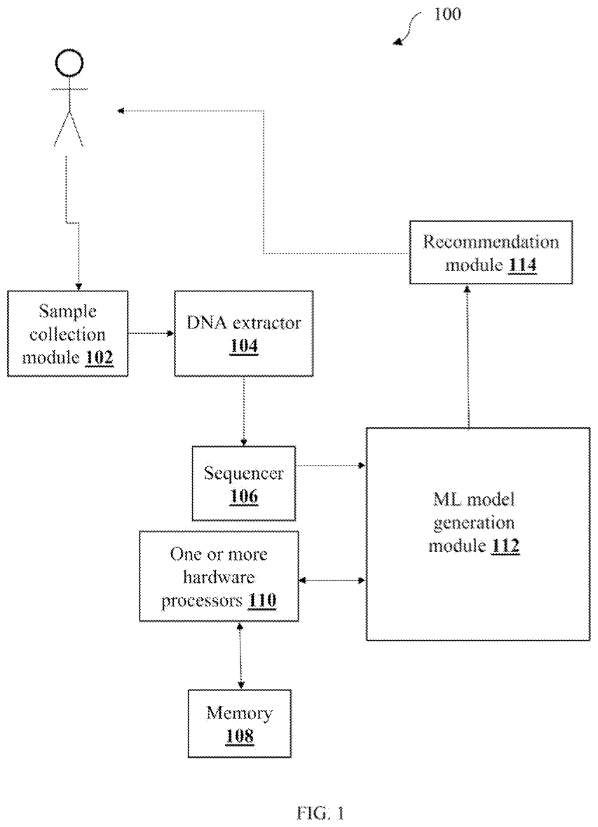

Referring now to the drawings, and more particularly to FIG. 1 through FIG. 4, where similar reference characters denote corresponding features consistently throughout the figures, there are shown preferred embodiments and these embodiments are described in the context of the following exemplary system and/or method.

According to an embodiment of the disclosure, a block diagram of the system 100 for identifying and utilizing a frugal set of markers for classification of a test biological sample is shown in FIG. 1. The system 100 consists of a sample collection module 102, a DNA extractor 104, a sequencer 106, a memory 108, one or more hardware processors (referred as a processor, herein after) 110, a machine learning model generation module 112, and a recommendation module 114 as shown in FIG. 1. The processor 110 is in communication with the memory 108.

It may be understood that the system 100 comprises one or more computing devices, such as a laptop computer, a desktop computer, a notebook, a workstation, a cloud-based computing environment and the like. It will be understood that the system 100 may be accessed through one or more input/output interfaces (not shown in Figures), collectively referred to as I/O interface or user interface. Examples of the I/O interface may include, but are not limited to, a user interface, a portable computer, a personal digital assistant, a handheld device, a smartphone, a tablet computer, a workstation and the like. The I/O interface are communicatively coupled to the system 100 through a network.

In an embodiment, the network may be a wireless or a wired network, or a combination thereof. In an example, the network can be implemented as a computer network, as one of the different types of networks, such as virtual private network (VPN), intranet, local area network (LAN), wide area network (WAN), the internet, and such. The network may either be a dedicated network or a shared network, which represents an association of the different types of networks that use a variety of protocols, for example, Hypertext Transfer Protocol (HTTP), Transmission Control Protocol/Internet Protocol (TCP/IP), and Wireless Application Protocol (WAP), to communicate with each other. Further, the network may include a variety of network devices, including routers, bridges, servers, computing devices, storage devices. The network devices within the network may interact with the system 100 through communication links.

The system 100 supports various connectivity options such as BLUETOOTH®, USB, ZigBee and other cellular services. The network environment enables connection of various components of the system 100 using any communication link including Internet, WAN, MAN, and so on. In an exemplary embodiment, the system 100 is implemented to operate as a stand-alone device. In another embodiment, the system 100 may be implemented to work as a loosely coupled device to a smart computing environment. The components and functionalities of the system 100 are described further in detail.

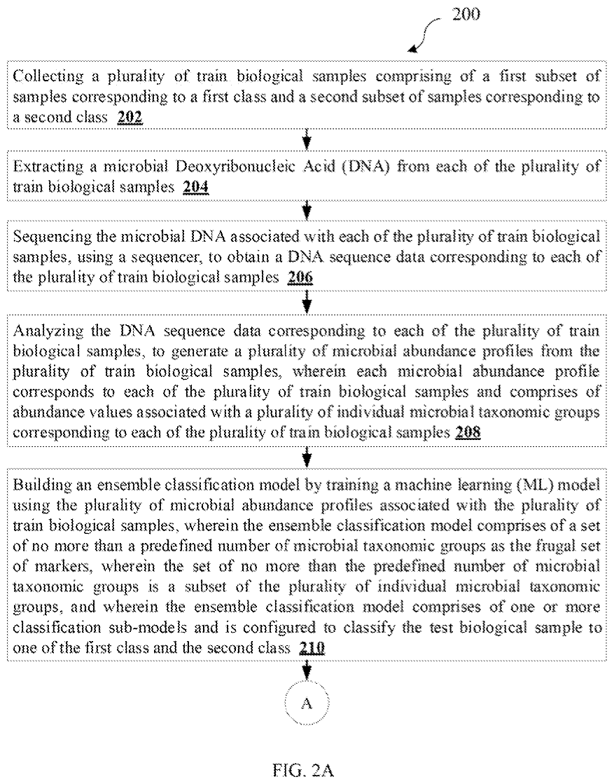

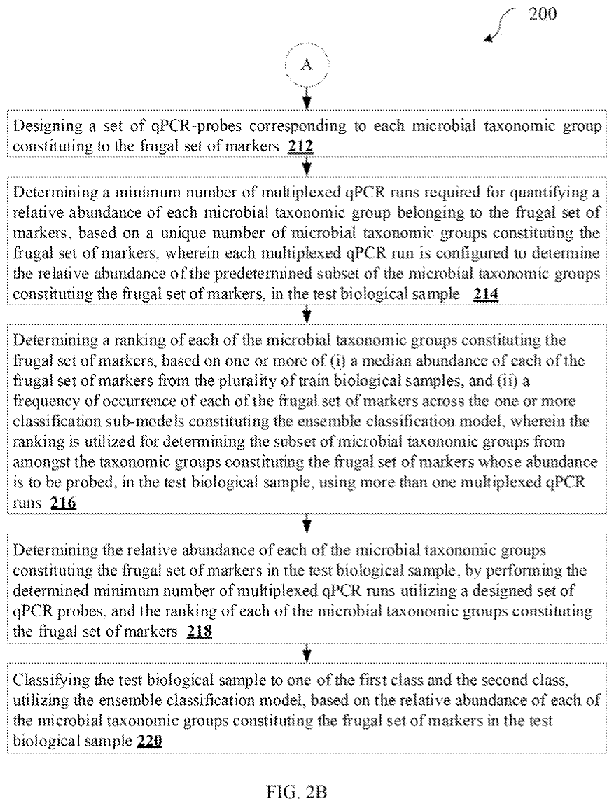

In operation, a flow diagram of a method 200 for identifying and utilizing a frugal set of markers for classification of a test biological sample, according to some embodiments of the present disclosure is shown in FIG. 2A-2B. The method 200 depicted in the flow chart may be executed by a system, for example, the system, 100 of FIG. 1. In an example embodiment, the system 100 may be embodied in a computing device.

Operations of the flowchart, and combinations of operation in the flowchart, may be implemented by various means, such as hardware, firmware, processor, circuitry and/or other device associated with execution of software including one or more computer program instructions. For example, one or more of the procedures described in various embodiments may be embodied by computer program instructions. In an example embodiment, the computer program instructions, which embody the procedures, described in various embodiments may be stored by at least one memory device of a system and executed by at least one processor in the system. Any such computer program instructions may be loaded onto a computer or other programmable system (for example, hardware) to produce a machine, such that the resulting computer or other programmable system embody means for implementing the operations specified in the flowchart. It will be noted herein that the operations of the method 200 are described with help of system 100. However, the operations of the method 200 can be described and/or practiced by using any other system.

Initially at step 202, the method 200 a plurality of train biological samples is collected through the sample collection module 102. Each train biological sample comprises at least one of a vaginal swab sample, a cervical mucus sample, a cervical swab sample, a vaginal swab including swab sample of a vaginal fornix, a urine sample, an amniotic fluid sample, a blood sample (whole blood sample), a serum sample, a plasma sample, a placental swab, an umbilical swab, a stool sample, a skin swab, an oral swab, a saliva sample, a periodontal swab, a throat swab, a nasal swab, a vesicle fluid sample, a nasopharyngeal swab, a nares swab, a conjunctival swab, a genital swab, a rectum swab, a tracheal aspirate, and a bronchial swab. The plurality of train biological sample comprises of a subset of samples corresponding to a first class and a remaining subset of samples corresponding to a second class. The biological samples refer to any type of samples sourced from animals, humans, plants etc. The first class and the second class refer to two different biological states that are exhibited by the individual/environment from where the sample is sourced/collected. For example, the first class may refer to an individual (or a group of individuals) in a healthy class and the second class may refer to an individual (or a group of individuals) in a diseased class.

Further at step 204, A microbial Deoxyribonucleic Acid (DNA) is extracted from each of the plurality of train biological samples, through the DNA extractor 104. At step 206, the extracted microbial DNA associated with each train biological sample is then sequenced through the sequencer 106 to obtain a DNA sequence data. The sequencer 106 amplifies and sequences either full-length or specific variable regions of the bacterial 16S rRNA or any other marker genes from the extracted microbial DNA, or any appropriate signature-/marker-gene sequences from the extracted microbial DNA, using a next-generation DNA sequencing (NGS) platform, or Oxford nanopore sequencing, or any other DNA sequencing technique and platform (including classical Sanger sequencing). In another implementation, alternate ways of characterizing/sequencing/measuring parameters of the train biological sample, such as whole genomics shotgun (WGS) metagenomic sequencing, proteomics, metabolomics, etc., can also be used.

At step 208 of the method 200, the DNA sequence data associated with each train biological sample is then analyzed to generate a microbial abundance profile. A plurality of microbial abundance profiles is generated from the plurality of train biological samples. Each microbial abundance profile associated with each train biological sample comprise abundance values associated with a plurality of individual microbial taxonomic groups present in each of the plurality of train biological samples.

At step 210 of the method 200, an ensemble classification model is built using the microbial abundance profiles generated from the plurality of train biological samples, through the ML model generation module 112. The ensemble classification model comprises of a set of no more than a predefined number of microbial taxonomic groups as the frugal set of markers. The set of no more than the predefined number of microbial taxonomic groups is a subset of plurality of individual microbial taxonomic groups. Further, the ensemble classification model comprises of one or more classification sub-models and is configured to classify a test biological sample to one of the first class and the second class.

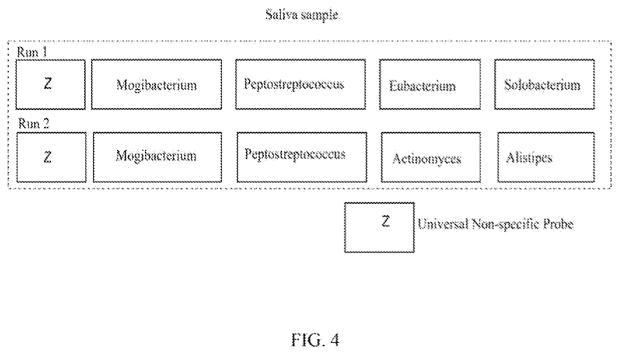

Further at step 212 of the method 200, a set of qPCR-probes corresponding to the microbial taxonomic groups constituting the frugal set of markers, is designed. FIG. 4 illustrates an exemplary multiplex qPCR design for detecting and quantifying microbial DNA having an exemplary set of qPCR-probes, according to some embodiments of the present disclosure. As shown in FIG. 4, the two sequential multiplex qPCR runs namely a first multiplex qPCR run (Run 1) and a second multiplex qPCR run (Run 2) are defined/performed for determining quantitative abundance of each of the microbial taxonomic groups (belonging to the frugal set of markers), whose DNA is targeted by the designed set of qPCR-probes. Each run of the first multiplex qPCR run (Run 1) and the second multiplex qPCR run (Run 2) includes five probes (hence it is also called as five-plex qPCR run) where each probe is utilized for detecting corresponding microbial taxonomic groups (constituting the frugal set of markers) each of the set of qPCR-probes, from the list having: Mogibacterium, Peptostreptococcus, Eubacterium, Solobacterium, Actinomyces, and Alistipe. Also, each run of the first multiplex qPCR run (Run 1) and the second multiplex qPCR run (Run 2) contains a universal non-specific probe (denoted as ‘Z’ in FIG. 4).

Design Configuration & Number of qPCR Runs Required for Quantifying the Abundance of Target Microbial Taxa/Features:

The relative abundance of each of the microbial taxonomic groups constituting the frugal set of markers, that are common to each of the minimum number of the multiplexed qPCR runs, is determined based on a normalizing factor (NFᵣᵤₙ) associated with each multiplexed qPCR run and the relative abundance of associated microbial taxonomic group in the corresponding multiplexed qPCR run.

Let us assume that a maximum of five unique DNA fragments, each representing a microbial taxa or spike DNA, can be quantified in a one multiplexed qPCR run. Therefore, to analyze a disease signature (captured in an ML model) comprising of ‘n’ microbial taxa/features, a minimum of (1+ ┌(n−4)/4┐) multiplexed qPCR runs would be required wherein ‘n’ is the unique number of microbial taxonomic groups constituting the frugal set of markers, and wherein each multiplexed qPCR run is configured to determine, in the test biological sample, the relative abundance of a predetermined subset of the microbial taxonomic groups constituting the disease signature. This minimum number is based on assumptions that:
(a) the spike DNA should be analyzed at least once in one of the ‘(1+ ┌(n=4)/4┐)’ multiplexed qPCR runs; and
(b) an overlap of at least one microbial taxa/features was done between two corresponding runs.

For example, if a disease signature comprises of 8 microbial taxa (A, B, C, D, E, F, G. and H), then at least TWO multiplexed qPCR runs would be required, where Z is the spike DNA of known concentration and taxa ‘D’ is analyzed in both multiplexed qPCR runs. Here, ┌(n−4)/4┐ indicates a ceiling value of the expression. Thus, the minimum no. of required qPCR runs would be:
1 for 1-4 signatures/features
2 for 5-8 signatures/features
3 for 9-12 signatures/features
4 for 13-16 signatures/features, and so on . . . .

### Example A: Run 1: Z A B C D; Run2: D E F G H

Similarly, for a feature size of 12 (A, B, C, D, E, F, G, H, I, J, K, and L), at least THREE multiplexed qPCR runs would be required, where Z is the spike DNA of known concentration and taxa ‘D’ and ‘H’ are analyzed in twice.

### Example B: Run 1: Z A B C D; Run 2: D E F G H; Run 3: H I J K L

If the number of features constituting the signature is not optimal for the above condition, i.e., for e.g., the number of features is 10, then more than one microbial taxon can be analyzed twice. The same is exemplified below, wherein taxa C and D are analyzed twice (in Runs 1 and 2). Similarly, taxa F and G are also analyzed twice (in Runs 2 and 3).

### Example C: Run 1: Z A B C D; Run 2: C D E F G; Run3: F G H I J

In alternate implementations, the spike DNA (Z) can be analyzed in each of the runs. In that scenario, the first multiplexed qPCR will be able to accommodate up to FOUR features. Each additional multiplexed qPCR run will accommodate up to THREE new/additional features as shown by underlining in the example below. Thus, two multiplexed qPCR runs would be required for a feature set of up to seven; three qPCR runs for a feature set of up to ten and so on.

### Run 1: Z A B C D: Run 2: Z D E F G; Run 3: Z G H I J

Furthermore, if the number of features is not optimal for the above condition, then two or more taxa/features can be analyzed multiple times as shown in example C.

Methodology to Interpret/Quantify the Abundance of a Microbial Taxon from Data Obtained from Above qPCR Configurations:

Given that the concentration of the spike DNA (Z) is previously known—say X₁. If the measured concentration of Z in the multiplexed qPCR is X₂, then all the measured concentration in a single multiplexed qPCR run can be normalized multiplying by a normalizing factor (NFᵣᵤₙ) of X₁/X₂.

In cases where the spike DNA is only analyzed in only one of the multiplexed qPCR runs (as shown in examples A, B and C), then the normalized values of the taxa/feature in the first run which is/are re-analyzed in the Run 2, can be used for adjusting the concentrations inferred from the Run 2 of the multiplexed qPCR. Following Example-A (described previously),
Actual conc of Z: X₁
Measured conc of Z: X₂
Normalizing factor NFᵣᵤₙ: X₁/X₂
Inferred conc. of A (from Run 1): A′ᵣᵤₙ₁×NFᵣᵤₙ₁
Inferred conc. of B (from Run 1): B′ᵣᵤₙ₁×NFᵣᵤₙ₁
Inferred conc. of C (from Run 1): C′ᵣᵤₙ₁×NFᵣᵤₙ₁
Inferred conc. of D (from Run 1): D′ᵣᵤₙ₁×NFᵣᵤₙ₁
   Where A′ᵣᵤₙ₁, B′ᵣᵤₙ₁, C′ᵣᵤₙ₁, and D′ᵣᵤₙ₁ are the measured/analyzed concentrations of taxa/feature A, B, C and D respectively.
   Normalizing factor NFᵣᵤₙ₂: Inferred conc. of D from Run 1/Measured concentrations of feature D in Run 2
Inferred conc. of E: E′ᵣᵤₙ₁×NFᵣᵤₙ₂
Inferred conc. of F: F′ᵣᵤₙ₁×NFᵣᵤₙ₂
Inferred conc. of G: G′ᵣᵤₙ₁×NFᵣᵤₙ₂
Inferred conc. of H: H′ᵣᵤₙ₁×NFᵣᵤₙ₂
   The same protocol may be repeated for normalizing/adjusting the concentrations measured from all subsequent runs (as in example B). In case wherein more than once feature is analyzed in subsequent runs (as in example C), a median Normalizing factor (NF)-derived from the NFs for each of the replication features may be used for computing the inferred concentrations from that run.

In alternate implementations, wherein the spike DNA (Z) is analyzed in each of the runs (as in example D), Normalizing factor (NF) corresponding to each of the runs may be computed and used for inferring the concentrations of the constituent features. In cases, where the measured spike DNA (Z) concentration varies by more than 25% from the actual concentration, it is suggested that the observations from the said multiplexed qPCR run be discarded, and a fresh multiplexed qPCR run for the sub-set of features be performed.

In an alternate implementation using multiplex qPCR runs, the marker feature having the lowest variance in relative abundance in training data across both the classes, is selected as the anchor marker (AM), and the relative abundance of each of the markers is computed by multiplying the ratio of their estimated/inferred DNA concentrations and the estimated/inferred DNA concentration of AM with the median abundance of AM across all training data. For example, if the marker features are A, B, C and D. wherein A is the anchor marker (AM) having a median abundance of ABN_{AM}, then the abundances of the marker features B, C and D will be computed as;

$${ABN}_{B} = ( Inferred conc . of B / Inferred conc . of A ) \times {ABN}_{AM} \\ {ABN}_{C} = ( Inferred conc . of C / Inferred conc . of A ) \times {ABN}_{AM} \\ {ABN}_{D} = ( Inferred conc . of D / Inferred conc . of A ) \times {ABN}_{AM}$$

Further at step 214, a minimum number of multiplexed qPCR runs required for quantifying the relative abundance of each microbial taxonomic group belonging to the frugal set of markers are determined based on a unique number of microbial taxonomic groups constituting the frugal set of markers. The minimum number is determined using the formula: 1+[(n−4)/4], where ‘n’ is the unique number of microbial taxonomic groups constituting the frugal set of markers, and wherein each multiplexed qPCR run is configured to determine, in the test biological sample, the relative abundance of a predetermined subset of the microbial taxonomic groups constituting the frugal set of markers.

At step 216 of the method 200, a ranking of the microbial taxonomic groups constituting the frugal set of markers is determined. The ranking is utilized for determining the subset of microbial taxonomic groups from amongst the taxonomic groups constituting the frugal set of markers whose abundance is to be probed, in the test biological sample, using more than one multiplexed qPCR runs. The ranking is determined based on one or more of (i) a median abundance of each of the frugal set of markers from the train biological samples, and (ii) a frequency of occurrence of each of the frugal set of markers across the one or more classification sub-models constituting the ensemble classification model. More specifically, the one or more predetermined microbial marker sequences (from amongst the set of predetermined microbial marker sequences) that has/have the highest (or relatively higher) median abundance or frequency of occurrence (as compared to the median abundance(s) or the frequency of occurrence of each microbial marker sequences in the remaining set of predetermined microbial marker sequences) across the plurality of training stool samples is/are common to the multiplex qPCR runs.

It should be appreciated that the collection of the plurality of training biological sample, building the ensemble model, and subsequently identifying the frugal set of biomarkers, through steps 202 to 216, are the one-time process.

At step 218 of the method 200, the relative abundance of each of the microbial taxonomic groups constituting the frugal set of markers is determined in the test biological sample by performing the determined minimum number of multiplexed qPCR runs utilizing the designed set of qPCR probes, and ranking of the microbial taxonomic groups constituting the frugal set of markers is designed.

And finally at step 220 of the method 200, the test biological sample is classified to one of the first class or the second class utilizing the ensemble classification model, and the determined relative abundance of the each of the microbial taxonomic groups constituting the frugal set of markers in the test biological sample.

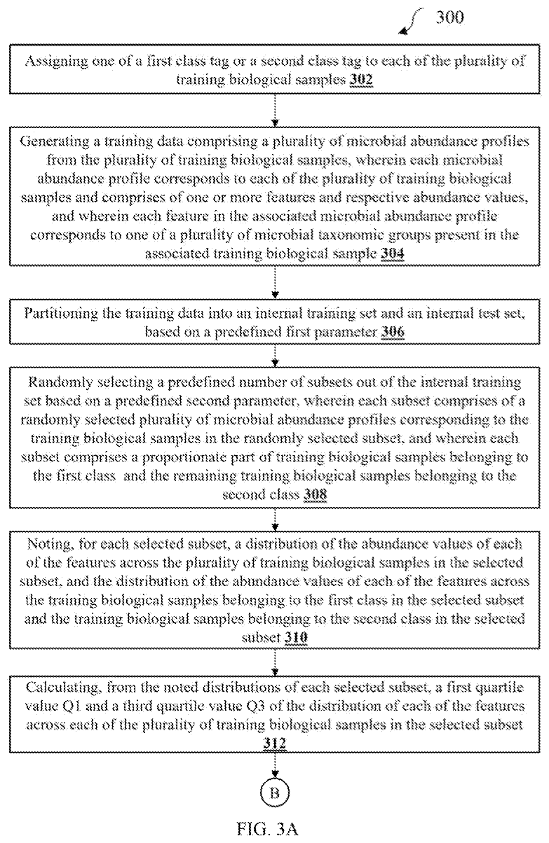

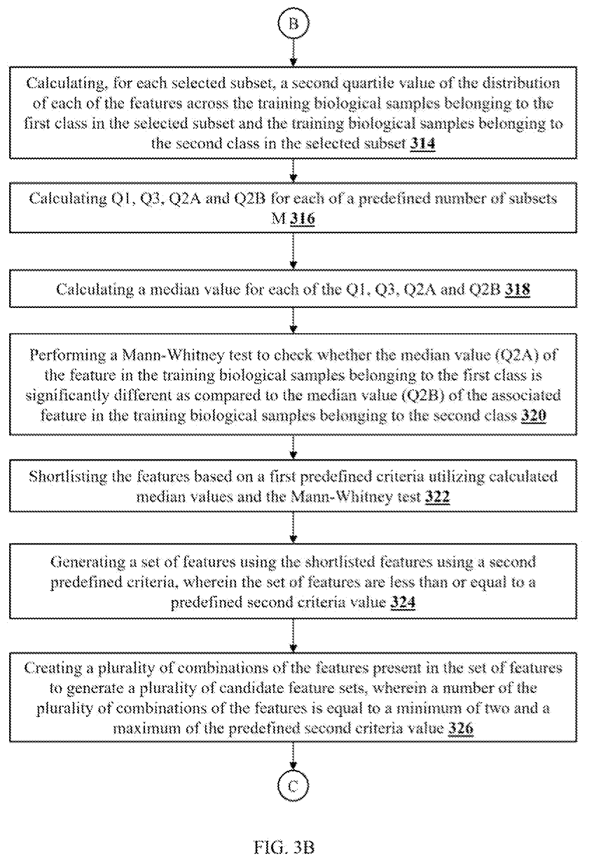

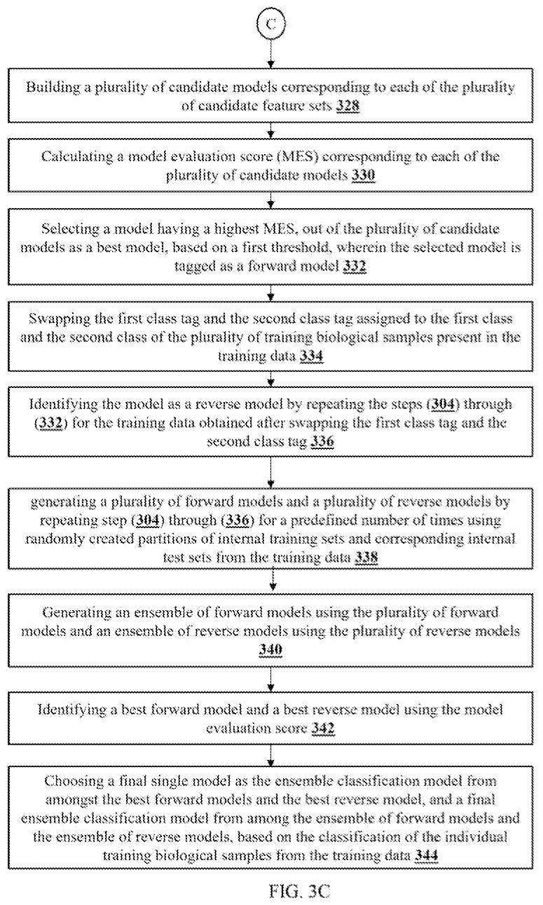

According to an embodiment of the disclosure, the ensemble classification model is built only one time. FIG. 3A-3C are flowcharts illustrating the steps involved in building an ensemble classification model, according to some embodiments of the present disclosure. The method for building the ensemble classification model accepts data in form of a feature table for multiple observations (or training biological samples) wherein each observation/training biological sample is defined by ‘N’ features (F) which are either or both of continuous and counted variables with (N≥1). In case of training data (TR), each of the training biological samples/observations further have a preassigned class/category/state which is binary in nature, i.e., the first class (A) (e.g., affiliating to healthy samples category) and the second class (B) (e.g., affiliating to unhealthy or disordered samples category). In case of test data (TS) or data received during actual deployment of the method, the model(s) built based on training data predicts the class/category/state of the training biological samples/observations. During training process, the following steps are followed:

Initially at step 302, a first class tag or a second class tag is assigned to each of the training biological sample in the plurality of training biological samples. At step 304, the training data comprising of a plurality of microbial abundance profiles corresponding to each of the plurality of training biological samples, is generated. Each microbial abundance profile is corresponding to each of the plurality of training biological sample and comprising of one or more features and respective abundance values of the features. Each feature in the microbial abundance profile corresponds to one of a plurality of microbial taxonomic groups present in the associated training biological sample. In the next step 306, the training data (TR) is randomly partitioned into two sets—namely, an internal-train (ITR) and an internal-test (ITS), based on a predefined first parameter ‘L₁’. Wherein, L₁% training biological samples from the total training data constitute the ITR set and (100−L₁) % of the training biological samples constitute the ITS set. Furthermore, the random partitioning into ITR and ITS sets is performed using a stratified sampling approach with the intent of preserving the relative proportion of training biological samples belonging to the first class (A) or the second class (B) in the total training data in these newly drawn subsets.

In the next step 308, a predefined number of subsets are randomly selected out of the internal training set based on a pre-defined second parameter (12). Each of the subset comprises a randomly selected plurality of microbial abundance profiles corresponding to the plurality of training biological samples in the randomly selected subset, and wherein each of the subset comprises a proportionate part of training biological samples belonging to the first class (A) and the remaining training biological samples belonging to the second class (B). Thus, from ITR, ‘M’ randomly drawn subsets ITRSᵢ (e.g., ITRS₁, ITRS₂, ITRS₃ . . . . ITRS_{M}), each containing S training biological samples are further generated, wherein S=L₂% of the training biological samples present in ITR. For example, the values of L₂ and M are 80% and 100 respectively and have also been used as default values during the validation experiments.

In the next step 310, for each selected subset, a distribution of the abundance values of each of the features across the plurality of training biological samples in the selected subset, and the distribution of the abundance values of each of the features across the training biological samples belonging to the first class (A) in the selected subset and the training biological samples belonging to the second class (B) in the selected subset are noted. Thus, from each subset ITRSᵢ (where i=1, 2, 3, . . . , M), wherein there are total S training biological samples, each of which are described by N features (Fⱼ) (where j=1, 2, 3, . . . , N), the distributions of each of the features (ITRSᵢDFⱼ) across S training biological samples are noted. Similarly, from each subset ITRSᵢ, wherein there are S_{A} training biological samples belonging to the first class (A) and S_{B} training biological samples belonging to the second class (B), each of the training biological samples being described by N features (Fⱼ: j=1, 2, 3, . . . , N), the distributions of each of the features (ITRSᵢD_{A}Fⱼ) across S_{A} training biological samples, and the distributions of each of the features (ITRSᵢD_{B}Fⱼ) across S_{B} training biological samples are noted.

In the next step 312, from the noted distributions of each selected subset, a first quartile value (Q1) and a third quartile value (Q3) of the distribution of each of the features is calculated across each of the plurality of training biological samples in the selected subset. In an example, the respective first quartile value (Q1) and the third quartile value (Q3) of ITRSᵢDFⱼ may also be referred as Q1ITRSᵢDFⱼ and Q3ITRSᵢDFⱼ respectively.

Furthermore, in the next step 314, for each selected subset, a second quartile value of the distribution of each of the features across the training biological samples belonging to the first class (Q2_{A}) in the selected subset and the training biological samples belonging to the second class (Q2_{B}) in the selected subset is calculated. Thus, in an example, the median value (in other words, the second quartile value) of (ITRSᵢD_{A}Fⱼ) is referred as Q2ITRSᵢD_{A}Fⱼ, and the median value of (ITRSᵢD_{B}Fⱼ) is referred as Q2ITRSᵢD_{B}Fⱼ.

In the next step 316, for the M subsets of ITRSⱼ, a total of M values for each of Q1ITRSᵢDFⱼ, Q3ITRSᵢDFⱼ, Q2ITRSᵢD_{A}Fⱼ, and Q2ITRSᵢD_{B}Fⱼ, are calculated. Further at step 318, a median value ( [symbol] ⱼ) is calculated for all calculated Q1, a median value ( [symbol] ⱼ) is calculated for all calculated Q3, a median value ( [symbol] ) is calculated for all calculated Q2_{A} and a median value ( [symbol] ) is calculated for all calculated Q2_{B}. Thus,

$$= median of \\ \{ Q 1 {ITRS}_{1} {DF}_{j} , Q 1 {ITRS}_{2} {DF}_{j} , Q 1 {ITRS}_{3} {DF}_{j} , … Q 1 {ITRS}_{M} {DF}_{j} \}$$

$$= median of \\ \{ Q 3 {ITRS}_{1} {DF}_{j} , Q 3 {ITRS}_{2} {DF}_{j} , Q 3 {ITRS}_{3} {DF}_{j} , … Q 3 {ITRS}_{M} {DF}_{j} \}$$

$$= median of \\ \{ Q 2 {ITRS}_{1} {D}_{A} {F}_{j} , Q 2 {ITRS}_{2} {D}_{A} {F}_{j} , Q 2 {ITRS}_{3} {D}_{A} {F}_{j} , … Q 2 {ITRS}_{M} {D}_{A} {F}_{j} \}$$

$$= median of \\ \{ Q 2 {ITRS}_{1} {D}_{B} {F}_{j} , Q 2 {ITRS}_{2} {D}_{B} {F}_{j} , Q 2 {ITRS}_{3} {D}_{B} {F}_{j} , … Q 2 {ITRS}_{M} {D}_{B} {F}_{j} \}$$

(where i=1, 2, 3, . . . , M; and j=1, 2, 3, . . . , N)

In the next step 320, a Mann-Whitney test is performed to test if a median value of the feature (Fⱼ) is significantly (p<0.1) different between the training biological samples belonging to the first class (S_{A}) and the training biological samples belonging to the second class (S_{B}) in each of the M randomly drawn subsets ITRSⱼ. Other statistical tests based on the nature of distribution (e.g., t-test for normal distribution), nature of sampling (e.g., Wilcoxon signed rank test for paired case and control training biological samples) or other methods of statistical comparison relevant for microbiome datasets (e.g., ALDEx2) can also be adopted.

In the next step 322, the features are shortlisted based on a first predefined criteria utilizing calculated median values and the Mann-Whitney test. The first predefined criteria comprises if a feature Fⱼ is observed to have significantly (p<0.1) different values in S_{A} compared to S_{B} in more than 70% of M subsets, and if [symbol] >=Q2ₘᵢₙ OR [symbol] >=Q2ₘᵢₙ (using a pre-defined ‘abundance’ threshold and Q2ₘᵢₙ threshold (example value is 0 as described in the case study). Fⱼ is added to a set of shortlisted features (SF).

In the next step 324, a set of features is generated using the shortlisted features (SF) using a predefined second predefined criteria. An exemplary value of the predefined second predefined criteria is 15. Hence, in an embodiment, the set of features are less than or equal to 15. If the number of shortlisted features (SF) obtained in previous step satisfies the criteria 1≤SF≤15, then the training process proceeds to model building with all the features in SF. If no shortlisted features (SF) are obtained in previous (i.e., SF<1) then following step is performed with all the features Fⱼ for evaluating the ability of the features, when considered independently, to distinguish between training biological samples belonging to the first class (A) and the second class (B). Similarly, if the number of shortlisted features (SF) obtained in previous step exceeds fifteen (SF>15) then following step is performed with all the shortlisted features (SF) for evaluating the ability of the features, when considered independently, to distinguish between training biological samples belonging to the first class (A) and the second class (B).

Steps for shortlisting the features in case of SF<1 or SF>15: For each of the features (obtained previously) taken individually, different threshold values are used to classify the training biological samples belonging to the set ITR, and the results are cumulated to construct a receiver operating characteristic curve (ROC curve) for each of the features. The area under the curve (AUC) of the ROC curve of any feature (AUC^{F}) is indicative of the utility of the feature to distinguish between training biological samples belonging to the first class (A) and the second class (B), and the same is computed for every feature. The shortlisted features (SF) set is modified to include only the top fifteen features from a list of features arranged in a descending order of the AUC^{F} values.

In the next step 326, a plurality of combinations of the features present in the set of features is created to generate corresponding plurality of candidate feature sets (CF), wherein the plurality of combinations of features comprises a minimum of one and a maximum of 15 features. By definition the maximum possible candidate feature sets that can be created in this process is K=2¹⁵−1=32767 (i.e., maximum value of K=32767).

In the next step 328, a plurality of candidate models is built corresponding to each of the plurality of candidate feature sets. At step 330, a model evaluation score (MES) is calculated corresponding to each of the plurality of candidate models. For each candidate feature set CFK, a corresponding candidate model CM_{K} is built and evaluated as mentioned in the steps mentioned below.

Steps for evaluating the candidate model:
Step 1: The values of the features F, constituting a candidate feature set defining the training biological samples in ITR are transformed to Fⱼ′ such that − [symbol] ⱼ, [symbol] ⱼ, [symbol] , and

[symbol]

$${F}_{j}^{′} = 0 … if {F}_{j} < {j}_{} \\ {F}_{j}^{′} = 1 … if {F}_{j} > {j}_{} \\ {F}_{j}^{′} = 0.5 … if {j}_{} = {j}_{} \\ {F}_{j}^{′} = … if {j}_{} < {F}_{j} < {j}_{}$$

Step 2: If for a feature Fⱼ, it is observed that [symbol] > [symbol] , then the feature Fⱼ is tagged as a ‘numerator’ feature and added to a set of numerator features Fₙᵤₘₑᵣₐₜₒᵣ. Else, feature Fⱼ is tagged as a ‘denominator’ feature and added to a set of denominator features F_{denominator}.
Step 3: Each candidate model (CM_{K}) is constituted as a simple ratio function given below—

$${CM}_{K} = \frac{\sum {F}_{numerator}}{\sum {F}_{denominator}} … when {F}_{numerator} > 0 and {F}_{denominator} > 0 or , \\ {CM}_{K} = \frac{\sum {F}_{numerator} + 1}{\sum {F}_{denominator} + 1} … when either {F}_{numerator} or {F}_{denominator} = 0$$

wherein, ΣFₙᵤₘₑᵣₐₜₒᵣ represents the sum of values of all numerator features for a particular training biological sample, and,
wherein, ΣF_{denominator} represents the sum of values of all denominator features for a particular training biological sample.
   For each of the features, a transformed value Fⱼ′ as obtained above is used in the candidate model equation.
Step 4: A candidate model c is used to generate candidate model scores (CMS_{K}) for each of the training biological samples in the set ITR. From the set of scores CMS_{K}, the top 10 percentile and bottom 10 percentile scores are removed as outliers and thereafter the maximum and minimum scores from the set CMS_{K} are noted as CMS_{K}_{ₘₐₓ} and CMS_{K}_{ₘᵢₙ} respectively.
Step 5: Considering each of the scores in the set CMS_{K} as a threshold (T), the model CM_{K} is used to (re) classify the training biological samples in the training set (ITR.) such that—
a training biological sample is classified into the first-class if CMS>=T
or a training biological sample is classified into the second-class if CMS<T
   and based on a comparison of these classifications and the true/original classes of the training biological samples, Matthew's correlation coefficients (MCC) for each of the thresholds are calculated, to evaluate how well each of the thresholds can distinguish between training biological samples between the first class (A) and the second class (B).
Step 6: The threshold (Tₘₐₓ) which provides the maximum absolute MCC value (|MCCₘₐₓ|) is noted. If |MCCₘₐₓ|<0.4 for a candidate model CM_{K}, then the candidate model is discarded from further evaluation. Else, the |MCCₘₐₓ| value is considered as the ‘train-MCC’ value (|MCCₜᵣₐᵢₙ|) for the model ITS and the model and its corresponding Tₘₐₓ threshold is used to classify the training biological samples in the internal-test set (ITS). In another implementation of the process, the MCCₘₐₓ threshold may not be applied for retaining the candidate model for subsequent evaluation. Before classifying the training biological samples in the ITS set, the values of features characterizing the training biological samples of the ITS set are transformed using the method mentioned in step 17 while using the earlier obtained values of [symbol] ⱼ, [symbol] ⱼ, [symbol] , and [symbol] from the ITR set.
Step 7: The classification results on the training biological samples from the ITS set are compared against the true/original classes of the training biological samples (with pre-assigned labels), and the MCC for the model CM_{K} and its corresponding Tₘₐₓ threshold on the ITS training biological samples is calculated (MCCₜₑₛₜ).
Step 8: A model evaluation score (MES) for candidate model CM_{K} is calculated as MES=|(MCCₜᵣₐᵢₙ+MCCₜₑₛₜ)|−((MCCₜᵣₐᵢₙ−MCCₜₑₛₜ)|

In the next step 332, the model CM_{K} is tagged as a “strong model” if all the features in the corresponding candidate feature set satisfies the Mann-Whitney test based shortlisting criteria described above. Otherwise, if any of the features in the corresponding feature set fails to satisfy the Mann-Whitney test, the model CM_{K} is tagged as a “weak model”.

Further, the above process is repeated for candidate models and respective MES scores are used to rank all the models. The best model is subsequently chosen based on the MES score. In case, there are more than one model with the best MES score, the best model is chosen based on the following criteria (in order of preference):
(a) the model with fewer number of features (i.e., based on a smaller candidate feature set) is chosen.
(b) the model with lower Tₘₐₓ (threshold value) is chosen.

Further, the best model obtained through above steps is tagged as a forward model (MD_{fwd}). The model MD_{fwd} additionally constitutes its corresponding Tₘₐₓ threshold, the CMS_{K}_{ₘₐₓ} and CMS_{K}_{ₘᵢₙ} values, and the [symbol] ⱼ, [symbol] ⱼ, [symbol] , and [symbol] values corresponding to the ITR set.

In the next step 334, the tags assigned to the first class (A) and the second class (B) of the plurality of training biological samples present in the training data are swapped. At step 336, the steps 304 through 332 are repeated to determine the best model are repeated after swapping the class labels (A<->B) for the entire training set (TR) to obtain a best model tagged as the reverse model (MDᵣₑᵥ). The model MDᵣₑᵥ additionally constitutes its corresponding Tₘₐₓ threshold, the CMS_{K}_{ₘₐₓ} and CMS_{K}_{ₘᵢₙ} values, and the [symbol] ⱼ, [symbol] ⱼ, [symbol] , and [symbol] values corresponding to the ITR set.

At step 338, a plurality of forward models and a plurality of reverse models are generated, by repeating step (304) through (336) for a predefined number of times using randomly created partitions of internal training sets and corresponding internal test sets from the training. The steps (304) through (336) are iterated ‘R’ times using multiple randomly partitioned ITR and ITS sets generated initially. After each iteration, (i) the features constituting the models MD_{fwd} and the models MDᵣₑᵥ obtained in the current iteration (r) are compared against, and if necessary, appended to, a set of unique features F_{unq} that consists of respective features constituting the MD_{fwd} and MDᵣₑᵥ obtained in earlier iterations (i.e., up to iteration r−1). After ‘R’ iterations, a plurality of forward models and a plurality of reverse models are generated for a predefined number of times using randomly partitioned internal training set and the internal test set. The iterations proceed while the value of R satisfies the following criteria—
(i) R≤Rₘₐₓ
(ii) (|F_{unq}| after iteration R)>(|F_{unq}| after iteration R−R_{unq})
(iii) |F_{unq}| after iteration no. R<=Fetₘₐₓ
   Wherein, Rₘₐₓ is a pre-defined parameter indicating the maximum number of iterations allowed;
   Rung is a pre-defined parameter indicating the maximum number of iterations allowed without any cumulative increase in the number of unique features |F_{unq}| in the models being generated in consecutive iterations; and
   Fetₘₐₓ is a pre-defined parameter indicating the maximum allowed value of |F_{unq}| (i.e., the no. of unique features cumulated through the iterative process).

In the next step 340, an ensemble of forward models is generated using the plurality of forward models and an ensemble of reverse models is generated using the plurality of reverse models. This is referred as an ensemble of forward models (ENS-MD_{fwd}) and an ensemble of reverse models (ENS-MDᵣₑᵥ).

At step 342, the best models from each of these ensembles, i.e., the best of the forward models (BMD_{fwd}) and the best of the reverse models (BMDᵣₑᵥ) respectively, are identified.

If all models in an ensemble are “weak models”, the best model from the ensemble (BMD) is chosen by ranking the models based on their model evaluation scores and associated criteria. Also, if an ensemble contains more than one “strong models”, then only those strong models are considered for ranking based on their model evaluation scores and associated criteria as mentioned above, and the best model from the ensemble (BMD) is thereby chosen. The associated criteria include: (i) the model with fewer number of features (i.e., based on a smaller candidate feature set) is chosen, (ii) the model with lower Tₘₐₓ (threshold value) is chosen.

In the next step 344, a final single model (FM_{single}) is chosen as the ensemble classification model from amongst the best forward model and the best reverse model based on how they classify the individual training biological samples from the training data (TR). Once the best models from each of the ensemble of forward models and the ensemble of reverse models, i.e., the best of the forward models (BMD_{fwd}) and the best of the reverse models (BMDᵣₑᵥ) are identified, the final single model (FM_{single}) is chosen from amongst BMD_{fwd} and BMDᵣₑᵥ based on how well they can classify the individual training biological samples from the entire training set (TR). The AUC value for ROC curves for each of these two models are computed based on the predicted model scores for the training set (TR) training biological samples and their pre-assigned classes. The model having the best AUC for ROC value is selected as the final single model (FM_{single}). If both BMD_{fwd} and BMDᵣₑᵥ have the same AUC value, BMD_{fwd} is chosen as FM_{single}.

In an alternate implementation FM_{single} can be chosen based whether BMD_{fwd} or BMDᵣₑᵥ obtains a higher MCC value while classifying the TR training biological samples. Once the FM_{single} model has been chosen, for classification of any test biological samples from a test set (TS) or any sample data received during actual deployment, the FM_{single} model is used after:
(a) appropriately transforming the features corresponding to the training biological sample being classified using the [symbol] ⱼ, [symbol] ⱼ, [symbol] , and [symbol] values corresponding to the FM_{single} model,
(b) limiting the model score between a maximum of CMS_{Kmax} and a minimum of CMS_{Kmin} values corresponding to the FM_{single} model, and
(c) classification based on the model score using its corresponding threshold Tₘₐₓ.

According to an embodiment of the disclosure, the ensemble of forward models (ENS-MD_{fwd}) and the ensemble of reverse models (ENS-MDᵣₑᵥ) are also evaluated for corresponding collective classification efficiencies using an ensemble model scoring. In the ensemble scoring method, each of the models (MD) constituting an ensemble (ENS) are used to generate a model score (MS) for each of the training biological samples from the entire TR set. For any specific training biological sample the values of the features corresponding to the training biological sample are appropriately transformed using the [symbol] ⱼ, [symbol] ⱼ, [symbol] , and [symbol] values corresponding to the model MD. The model scores (MS) are then transformed into scaled model scores (SMS) having values between −1 and +1, using the following procedure:

$$SMS = ( MS - {T}_{max} ) / ( {CMS}_{{K}_{max}} - {T}_{max} ) , … when MS >= {T}_{max} , and \\ SMS = ( MS - {T}_{max} ) / ( {T}_{max} - {CMS}_{{K}_{min}} ) , … when MS < {T}_{max} ,$$

Wherein, Tₘₐₓ, CMS_{K}_{ₘₐₓ}, and CMS_{K}_{ₘᵢₙ} values corresponding to the respective model is used.

Let SMS_{avg} be the average of all SMS obtained using all models in ENS for a particular training biological sample.

When using Forward model [ENS-MD_{fwd}],

$${SMS}_{avg} = {SMS}_{avg}^{*} ( + 1 )$$

If SMS_{avg}>=0, training biological sample is classified as ‘B’
If SMS_{avg}<0, training biological sample is classified as ‘A’
When using Reverse model [ENS-MDᵣₑᵥ];
SMS_{avg}=SMS_{avg}*(−1) If SMS_{avg}>0, training biological sample is classified as ‘B’
If SMS_{avg}<=0, training biological sample is classified as ‘A’

If all models in one of the ensembles are weak models, then the other one having (one or more) strong models is selected as a final ensemble model (FMₑₙₛ), and subsequently used for classification of any test biological samples from the test set (TS) or any sample data received during actual deployment of the method, using the scoring and classification process mentioned in above paragraph. If both ensembles have constituent strong models, then both the ensembles are evaluated for their efficiency by scoring them on all individual training biological samples in TR. The AUC value for ROC curves for each of these two ensembles are computed based on the predicted SMS_{avg} for all the training set (TR) training biological samples and their pre-assigned classes. The ensemble of models having the best AUC for ROC value is selected as the final ensemble model (FMₑₙₛ). In case both ENS-MD_{fwd} and ENS-MDᵣₑᵥ exhibit equal AUC values then ENS-MD_{fwd} is chosen as the final ensemble model (FMₑₙₛ). In an alternate implementation, FMₑₙₛ can be chosen based whether ENS-MD_{fwd} and ENS-MDᵣₑᵥ obtains a higher average MCC value for their respective constituent models while classifying the TR training biological samples.

Thus, either the FM_{single} model or FMₑₙₛ ensemble of models can be used for classification of any test biological samples from a test set (TS) or any sample data received during actual deployment.

According to an embodiment of the disclosure, the system 100 comprises the recommendation module 114. The recommendation module 114 generates a feature set that can potentially be targeted/modulated for therapeutic purposes, based on the diagnosed feature profile(s). The recommendation module 114 is configured to design/recommend a pre-biotic or pro-biotic formulation which can modulate the taxonomic abundances of a microbiome in a diseased individual towards a control-like distribution.

According to an embodiment of the disclosure, the method for designing/recommending a recommendation with a personalized therapeutic formulation to the individual (in instances where the ML model classifies a test sample to a ‘disease’ class) is explained as follows:

At step 1, pair-wise correlations (using the Pearson's and/or spearman's correlation index) are computed between abundances of features (i.e., organisms/taxa/microbes) constituting the ML model and the abundances corresponding to the complete set of microbes computed individually from (a) the subset of biological samples corresponding to the healthy class i.e. the class of samples that were taken from individuals not having disease, and (b) the diseased class i.e. the class of samples that were taken from individuals having disease). Wherein the samples belonging to the healthy and diseased classes are used as training data for generating the ML model.

At step 2, positive and negative interactions between features (i.e., organisms/taxa/microbes) constituting the ML model and all other taxa in the healthy and the diseased class of training samples (individually) are deduced using critical correlation (r) value as the cut-off (as taught in Batushansky et al., 2016), such that inter-taxa correlation index values greater than +r value are affiliated as ‘positive interactions’, while those less than −r value are affiliated as ‘negative interactions’.

At step 3, the steps 1 and 2 are repeated 1000 times and only those interactions are considered relevant that appear in at least 70% of iterations with a BH (Benjamini-Hochberg) corrected p-value cut-off of 0.1 are retained (hereafter referred to as model taxa interactions corresponding to health and diseased class of samples).

At step 4, thereafter, following set of rules (indicated in Table 1 below) are used to arrive at the relevant candidate using the retained model taxa interactions:

TABLE 1

| Probiotic Candidates | Antibiotic Candidates |
|---|---|
| (M_{H} − C_{T})_{HP} && (M_{H} − C_{T})_{DP} | (M_{H} − C_{A})_{HN} ∥ (M_{H} − C_{A})_{DN} |
| (M_{H} − C_{T})_{DP} | (M_{D} − C_{A})_{HP} ∥ (M_{D} − C_{A})_{DP} |
| (M_{D} − C_{T})_{HN} && (M_{D} − C_{T})_{DN} | |

From Table 1,
M_{H} represents a model taxon having significantly higher abundance in healthy class;
M_{D} represents a model taxon having significantly higher abundance in diseased (unhealthy) class;
C_{T} represents a potential therapeutic candidate;
C_{A} represents a potential antibiotic target candidate;
M_{H}-C_{T} represents an interaction between a model taxon (abundant in healthy class) with a potential therapeutic candidate;
M_{D}-C_{T} represents an interaction between a model taxon (abundant in diseased class) with a potential therapeutic candidate;
M_{D}-C_{A} represents an interaction between a model taxon (abundant in diseased class) with a potential antibiotic target candidate;
MH-C_{A} represents an interaction between a model taxon (abundant in healthy class) with a potential antibiotic target candidate;
HP represents a positive interaction in a healthy environment population;
HN represents a negative interaction in a healthy environment population;
DP represents a positive interaction in a diseased environment population; and
DN represents a negative interaction in a diseased environment population.

According to an embodiment of the present disclosure, the method 200 can also be explained with the help of experimental results. The method for identification of a frugal set of combination of biomarkers has been tested with clinical datasets pertaining to two diseases, i.e., breast cancer and PCOS. The results of these two datasets have been provided below.

Case study 1: Raw sequenced reads were taken from the publicly available breast cancer study. The raw sequenced data were first quality filtered using Prinseq-lite tool and taxonomic affiliations of the reads in the sample were then determined using Ribosomal Database Project version 2.12. Finally, the abundance table was generated for estimating the abundance of each taxonomic feature in every sample. The raw abundance values were then rarefied at a minimum sampling depth/rarefaction depth (i.e., minimum library size determined after the sequencing run) using ‘qiime feature-table rarefy’ function in qiime tools of qiime2 package. This was done in order to remove any bias in the dataset and rarefied values were then further used for model generation.

Predicting breast cancer from the above-mentioned data (Stool microbiome; Total number of samples=94; Cases=47; Controls=47). The classification models were built using both the methodology described in the present disclosure and Random Forest (RF) methodology for comparison. Table 1 shows evaluation results for 100 Models built with Train-Test sets which were partitioned in 90:10 proportion by stratified random selection of samples, where AUC depicts Area Under Curve and MCC depicts Matthew's correlation coefficient.

| | Mean | | | | |
|---|---|---|---|---|---|
| | Feature | | | | |
| | Count | | Std. | | Std. |
| Breast | in the | Mean | Dev | Mean | Dev |
| Cancer | model | AUC | AUC | MCC | MCC |
| RF (all | 201 | 0.72 | 0.16 | 0.31 | 0.31 |
| features) | | | | | |
| RF (15 | 15 | 0.71 | 0.14 | 0.31 | 0.31 |
| features) | | | | | |
| ENRICH | 4 | 0.73 | 0.15 | 0.36 | 0.25 |
| (single) | | | | | |
| ENRICH | 16 | 0.73 | 0.14 | 0.35 | 0.25 |
| (ensemble) | | | | | |

Table 1 shows evaluation results for 100 Models built with Train-Test sets which were partitioned in 90:10 proportion by stratified random selection of samples

Case study 2: Raw sequenced reads were taken from the publicly available PCOS study. The raw sequenced data were first quality filtered using Prinseq-lite tool and taxonomic affiliations of the reads in the sample were then determined using Ribosomal Database Project version 2.13. Finally, the abundance table was generated for estimating the abundance of each taxonomic feature in every sample. The raw abundance values were then percent normalized to remove any bias in the dataset and were then further used for model generation.

Predicting PCOS from the above-mentioned data (Salivary microbiome; Total number of samples=43; Cases=24; Controls=19). The classification models were built using both the methodology described in the present disclosure and Random Forest for comparison. The table 2 shows evaluation results for 100 Models built with Train-Test sets which were partitioned in 90:10 proportion by stratified random selection of samples.

| | Mean | | Std. | | Std. |
|---|---|---|---|---|---|
| | Feature | Mean | Dev | Mean | Dev |
| PCOS | Count | AUC | AUC | MCC | MCC |
| RF (all | 194 | 0.74 | 0.22 | 0.36 | 0.41 |
| features) | | | | | |
| RF (15 | 15 | 0.74 | 0.21 | 0.42 | 0.28 |
| features) | | | | | |
| ENRICH | 3 | 0.75 | 0.17 | 0.41 | 0.26 |
| (single) | | | | | |
| ENRICH | 8 | 0.78 | 0.18 | 0.42 | 0.28 |
| (ensemble) | | | | | |

The table 2 shows evaluation results for 100 Models built with Train-Test sets which were partitioned in 90:10 proportion by stratified random selection of samples.

According to an embodiment of the disclosure, the system 100 can also be explained with the help of experimental workflow for a woman for breast cancer risk screening.
Step 1: Obtain a stool sample from an individual for whom breast cancer risk screening is intended.
Step 2: Quantify the raw abundances of various microbial taxonomic groups in the stool sample. Methodology for this involves extraction of microbial DNA contents from the collected stool sample followed by amplification and sequencing of either full-length or specific variable regions of the bacterial 16S rRNA marker genes using a next-generation sequencing platform or by using the multiplex qPCR-based quantification methodology. In either case, the sequencing depth (i.e., number of reads obtained by sequencing the microbial DNA content of the stool sample) obtained should exceed a pre-defined threshold, wherein the said threshold refers to the rarefaction depth that needs to be determined and/or applied to adjust for differences in library sizes across samples in current the sequencing run or for removing the sequencing bias).

TABLE 3
Raw abundance of features in a test sample

| Features | Raw Abundance |
|---|---|
| Butyricicoccus | 159 |
| Clostridium_IV | 95 |
| Clostridium_sensu_stricto | 0 |
| Faecalibacterium | 5936 |
| Flavonifractor | 40 |
| Gemmiger | 465 |
| Lachnospiracea_incertae_sedis | 899 |
| Murimonas | 1 |
| Oscillibacter | 148 |
| . . . | |
| . . . | |
| Ruminococcus | 329 |
| Sporobacter | 0 |

Step 3: Rarefy the abundances of various taxa employing rarefaction depth i.e., minimum library size determined after the sequencing run wherein, rarefaction was done using ‘qiime feature-table rarefy’ function in qiime tools of qiime2 package.

TABLE 4
Rarefied abundance of features in the test sample

| | Rarefied |
|---|---|
| Features | Abundance |
| Butyricicoccus | 50 |
| Clostridium_IV | 34 |
| Clostridium_sensu_stricto | 0 |
| Faecalibacterium | 2221 |
| Flavonifractor | 10 |
| Gemmiger | 165 |
| Lachnospiracea_incertae_sedis | 306 |
| Murimonas | 0 |
| Oscillibacter | 46 |
| . . . | |
| . . . | |
| Ruminococcus | 131 |
| Sporobacter | 0 |

Step 4: From the rarefied abundance table 4, retain abundances of only the subset of taxa which overlap with the list of three taxa that are provided against ‘Single Best Training Model’ as mentioned below in Table 5.

TABLE 5
Model characteristics of features in the Single Best Training Model

| Features | Murimonas | Clostridium_sensu_stricto | Lachnospiracea_incertae_sedis |
|---|---|---|---|
| Q1 | 0 | 1 | 209.5 |
| Q3 | 1 | 5 | 436.5 |
| Q2_{A} | 0 | 0 | 336 |
| Q2_{B} | 1 | 3 | 252 |
| Numerator/ | Numerator | Numerator | Denominator |
| Denominator | | | |
| Feature | | | |
| Min Model | 0.5 | | |
| Score | | | |
| Max Model | 5.321305 | | |
| Score | | | |

| Threshold | 1.402786 |
|---|---|
| Model Type | Reverse |

As an example, assume that the three taxa in the taxonomic abundance profile obtained by processing the stool sample (in the manner mentioned in Steps 1 and 2) had the following rarefied abundances:
Abundance of Murimonas (i.e., feature 1 in training model) in collected stool sample: 0.000000
Abundance of Clostridium_sensu_stricto (i.e., feature 2 in training model) in collected stool sample: 0.000000
Abundance of Lachnospiracea_incertae_sedis (i.e., feature 3 in training model) in collected stool sample: 306.000000
Step 5: Using Q1 and Q3 values corresponding to each training model feature in the single best model (as mentioned in Table 5), and applying the transformation as mentioned above to the rarefied abundances, results in the following:
Transformed abundance (FMurimonas): 0.000000
Transformed abundance (FClostridium_sensu_stricto): 0.000000
Transformed abundance (FLachnospiracea_incertae_sedis): 0.425110

The transformed abundance of individual features as obtained above are then used appropriately in the candidate model equation (CM_{K}) (as replicated below), and numerator and denominator sums are computed. In this case, the values obtained are as follows: Since Numerator sum=0 and Denominator sum=0.425110 in this case, a value of 1 is added to both numerator and denominator.

$${CM}_{K} = \frac{\sum {F}_{numerator}}{\sum {F}_{denominator}} … when {F}_{numerator} > 0 and {F}_{denominator} > 0 or , \\ {CM}_{K} = \frac{\sum {F}_{numerator} + 1}{\sum {F}_{denominator} + 1} … when either {F}_{numerator} or {F}_{denominator} = 0$$

Numerator sum: 1.000000
Denominator sum: 1.425110
Step 6: The sample model score (MS) is computed next using above Numerator sum and Denominator sum. The sample model score (MS) is then transformed into scaled model score (SMS) (having values between −1 and +1, using following rules:

$$SMS = ( MS - {T}_{max} ) / ( {CMS}_{{K}_{max}} - {T}_{max} ) , … when MS >= {T}_{max} , and \\ SMS = ( MS - {T}_{max} ) / ( {T}_{max} - {CMS}_{{K}_{min}} ) , … when MS < {T}_{max} ,$$

Wherein, Tₘₐₓ, CMS_{K}_{_{max′}} and CMS_{K}_{ₘᵢₙ} values corresponding to the respective model is used.
For this purpose, the values of threshold: 1.402786,
Maximum model score: 5.321305, and Minimum model score: 0.500000 for single best model are employed.
Model score (MS): 0.701700
Scaled model score (SMS): −0.776580
Step 7: The SMS is then used for predicting the category of risk for the individual from whom the stool sample was obtained i.e., healthy or ‘at-risk for breast cancer’. Since both forward model and reverse model are evaluated. Here in this case, final selected single best model is a reverse model, hence the final prediction score value is calculated as (SMS*−1)
Final pred_score is 0.776580.
Since the value is >0, the prediction class is “B” i.e., Risk Category is “Breast Cancer”

Following the same series of steps, if the value of SMS is less than 0 then the prediction class will be “A” and the category of risk for the individual from whom the stool sample was obtained will be “Healthy”.
Step 8: Similarly, for Ensemble model, all the steps are repeated for all the single models in the ensemble and finally mean of all the Final prediction score calculated using sample model scores (SMS) and the class prediction is done based on final mean prediction score obtained.

The embodiments of present disclosure herein address unresolved problem of lengthy and time taking process of designing the artificially structured materials. The embodiment thus provides a method and system for designing artificially structured materials with customized functionalities. The method is faster and computationally less expensive than standard metamaterial simulations used in the prior art.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an.” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A method for identifying and utilizing a frugal set of markers for classification of a test biological sample, the method comprising:
collecting a plurality of train biological samples comprising of a first subset of samples corresponding to a first class and a second subset of samples corresponding to a second class;
extracting a microbial Deoxyribonucleic Acid (DNA) from each of the plurality of train biological samples;
sequencing the microbial DNA associated with each of the plurality of train biological samples, using a sequencer, to obtain a DNA sequence data corresponding to each of the plurality of train biological samples;
analyzing, via one or more hardware processors, the DNA sequence data corresponding to each of the plurality of train biological samples, to generate a plurality of microbial abundance profiles from the plurality of train biological samples, wherein each of the plurality of microbial abundance profiles corresponds to each of the plurality of train biological samples and comprises of abundance values associated with a plurality of individual microbial taxonomic groups corresponding to each of the plurality of train biological samples;
building, via the one or more hardware processors, an ensemble classification model by training a machine learning (ML) model using the plurality of microbial abundance profiles associated with the plurality of train biological samples, wherein the ensemble classification model comprises of a set of no more than a predefined number of microbial taxonomic groups as the frugal set of markers, wherein the set of no more than the predefined number of microbial taxonomic groups is a subset of the plurality of individual microbial taxonomic groups, and wherein the ensemble classification model comprises of one or more classification sub-models and is configured to classify the test biological sample to one of the first class and the second class;
designing, via the one or more hardware processors, a set of quantitative polymerase chain reaction (qPCR)-probes corresponding to each microbial taxonomic group constituting to the frugal set of markers;
determining, via the one or more hardware processors, a minimum number of multiplexed qPCR runs required for quantifying a relative abundance of each microbial taxonomic group belonging to the frugal set of markers, based on a unique number of microbial taxonomic groups constituting the frugal set of markers, wherein each multiplexed qPCR run is configured to determine the relative abundance of the predetermined subset of the microbial taxonomic groups constituting the frugal set of markers, in the test biological sample;
determining, via the one or more hardware processors, a ranking of each of the microbial taxonomic groups constituting the frugal set of markers, based on one or more of (i) a median abundance of each of the frugal set of markers from the plurality of train biological samples, and (ii) a frequency of occurrence of each of the frugal set of markers across the one or more classification sub-models constituting the ensemble classification model, wherein the ranking is utilized for determining the subset of microbial taxonomic groups from amongst the taxonomic groups constituting the frugal set of markers whose abundance is to be probed, in the test biological sample, using more than one multiplexed qPCR runs;
determining, via the one or more hardware processors, the relative abundance of each of the microbial taxonomic groups constituting the frugal set of markers in the test biological sample, by performing the determined minimum number of multiplexed qPCR runs utilizing a designed set of qPCR probes, and the ranking of each of the microbial taxonomic groups constituting the frugal set of markers; and
classifying, via the one or more hardware processors, the test biological sample to one of the first class and the second class, utilizing the ensemble classification model, based on the relative abundance of each of the microbial taxonomic groups constituting the frugal set of markers in the test biological sample.

2. The method of claim 1, wherein the minimum number of multiplexed qPCR runs is determined using a formula: 1+┌(n−4)/4┐, where n is the unique number of microbial taxonomic groups constituting the frugal set of markers.

3. The method of claim 1, wherein the ensemble classification model is built by:
(i) assigning one of a first class tag or a second class tag to each of the plurality of training biological samples;
(ii) generating a training data comprising a plurality of microbial abundance profiles from the plurality of training biological samples, wherein each microbial abundance profile corresponds to each of the plurality of training biological samples and comprises of one or more features and respective abundance values, and wherein each feature in the associated microbial abundance profile corresponds to one of a plurality of microbial taxonomic groups present in the associated training biological sample;
(iii) partitioning the training data into an internal training set and an internal test set, based on a predefined first parameter;
(iv) randomly selecting a predefined number of subsets out of the internal training set based on a predefined second parameter, wherein each subset comprises of a randomly selected plurality of microbial abundance profiles corresponding to the training biological samples in the randomly selected subset, and wherein each subset comprises of a proportionate part of training biological samples belonging to the first class and the remaining training biological samples belonging to the second class;
(v) noting, for each selected subset, a distribution of the abundance values of each of the features across the plurality of training biological samples in the selected subset, and the distribution of the abundance values of each of the features across the training biological samples belonging to the first class in the selected subset and the training biological samples belonging to the second class in the selected subset;
(vi) calculating, from the noted distributions of each selected subset, a first quartile value Q1 and a third quartile value Q3 of the distribution of each of the features across each of the training biological samples in the selected subset;
(vii) calculating, for each selected subset, a second quartile value of the distribution of each of the features across the training biological samples belonging to the first class Q2_{A} in the selected subset and the training biological samples belonging to the second class Q2_{B} in the selected subset;
(viii) calculating Q1, Q3, Q2_{A} and Q2_{B} for each of a predefined number of subsets M;
(ix) calculating a median value for each of the Q1, Q3, Q2_{A} and Q2_{B};
(x) performing a Mann-Whitney test to check whether the median value (Q2_{A}) of the feature in the training biological samples belonging to the first class is significantly different as compared to the median value (Q2_{B}) of the associated feature in the training biological samples belonging to the second class;
(xi) shortlisting the features based on a first predefined criteria utilizing calculated median values and the Mann-Whitney test;
(xii) generating a set of features using the shortlisted features using a second predefined criteria, wherein the set of features are less than or equal to a predefined second criteria value;
(xiii) creating a plurality of combinations of the features present in the set of features to generate a plurality of candidate feature sets, wherein a number of the plurality of combinations of the features is equal to a minimum of two and a maximum of the predefined second criteria value;
(xiv) building a plurality of candidate models (CM_{K}) corresponding to each of the plurality of candidate feature sets;
(xv) calculating a model evaluation score (MES) corresponding to each of the plurality of candidate models;
(xvi) selecting a model having a highest MES, out of the plurality of candidate models as a best model, based on a first threshold (Tₘₐₓ), wherein the selected model is tagged as a forward model;
(xvii) swapping the first class tag and the second class tag assigned to the first class and the second class of the plurality of training biological samples present in the training data;
(xviii) identifying and subsequently tagging the model as a reverse model by repeating the steps (ii) through (xvi) for the training data obtained after swapping the first class tag and the second class tag;
(xix) generating a plurality of forward models and a plurality of reverse models by repeating step (ii) through (xviii) for a predefined number of times using randomly created partitions of internal training sets and corresponding internal test sets from the training data;
(xx) generating an ensemble of forward models (ENS-MD_{fwd}) using the plurality of forward models and an ensemble of reverse models (ENS-MDᵣₑᵥ) using the plurality of reverse models;
(xxi) identifying a best forward model and a best reverse model using the model evaluation score; and
(xxii) choosing a final single model (FM_{single}) from amongst the best forward models and the best reverse model, and a final ensemble classification model (FMₑₙₛ) from among the ensemble of forward models and the ensemble of reverse models, based on the classification of the individual training biological samples from the training data.

4. The method of claim 3, further comprises:
classifying each of the set of shortlisted features using a second threshold value different from the first threshold (Tₘₐₓ); and
cumulating the results to construct a receiver operating characteristic curve (ROC) for each of the shortlisted features, wherein an area under the curve (AUC) of the ROC is indicative of utility of the feature to distinguish between the training biological samples belonging to the first class and the second class.

5. The method of claim 3, wherein calculating the model evaluation score (MES) comprises:
transforming the values of the set of features as follows:
$${F}_{j}^{′} = 0 … if {F}_{j} < {j}_{} \\ {F}_{j}^{′} = 1 … if {F}_{j} > {j}_{} \\ {F}_{j}^{′} = 0.5 … if {j}_{} = {j}_{} \\ {F}_{j}^{′} = … if {j}_{} < {F}_{j} < {j}_{} ;$$
collating the features out of the set of features as a set of numerator features (Fₙᵤₘₑᵣₐₜₒᵣ) if [symbol] > [symbol] , else, collating the features out of the set of features as a set of denominator features (F_{denominator});
constituting a ratio function for each of the candidate model as:
$${CM}_{K} = \frac{\sum {F}_{numerator}}{\sum {F}_{denominator}} … when {F}_{numerator} > 0 and {F}_{denominator} > 0 or , \\ {CM}_{K} = \frac{\sum {F}_{numerator} + 1}{\sum {F}_{denominator} + 1} … when either {F}_{numerator} or {F}_{denominator} = 0$$
wherein, ΣFₙᵤₘₑᵣₐₜₒᵣ represents the sum of values of all numerator features for a particular training biological sample, and,
wherein, ΣF_{denominator} represents the sum of values of all denominator features for a particular training biological sample;
generating a candidate model score (CMS_{K}) for each of the training biological samples in the internal train set;
removing the top 10 percentile and bottom 10 percentile scores as outliers from the set of scores CMS_{K}, and identifying maximum and minimum scores from the set CMS_{K} as CMS_{K}_{ₘₐₓ} and CMS_{K}_{ₘᵢₙ} respectively;
reclassifying the training biological samples in the internal train set by considering each score in the set CMS_{K} as threshold, such that the training biological sample is classified into the second class B if CMS_{K} is more than or equal to the threshold, or the training biological sample is classified into the first class if CMS_{K} is less than the threshold;
calculating Matthew's correlation coefficients (MCC) for each of the thresholds based on a comparison of the reclassified training biological sample and the original classes of the training biological samples, to evaluate how well each of the thresholds are able to distinguish between the training biological samples associated to the first class and the second class;
identifying the threshold as a first threshold (Tₘₐₓ) which provides maximum absolute MCC value;
discarding the candidate models for further evaluation if the maximum absolute MCC value is less than 0.4;
considering the (|MCCₘₐₓ|) value as the ‘train-MCC’ value (MCCₜᵣₐᵢₙ) for the model CM_{K} and the model and the first threshold (Tₘₐₓ) is used to classify the training biological samples in the internal-test set;
comparing the classification results on the training biological samples from the internal test set against the original classes of the training biological samples with pre-assigned labels, and the MCC for the model CM_{K} and the threshold Tₘₐₓ threshold on the internal train set is calculated (MCCₜₑₛₜ); and
calculating a model evaluation score (MES) for candidate model CM_{K} as: MES=|(MCCₜᵣₐᵢₙ+MCCₜₑₛₜ)|−|(MCCₜᵣₐᵢₙ−MCCₜₑₛₜ).

6. The method of claim 3, further comprising evaluating collective classification efficiencies of the ensemble of forward models (ENS-MD_{fwd}) and the ensemble of reverse models (ENS-MDᵣₑᵥ), using an ensemble model scoring method, wherein a model scores (MS) corresponding to each of the ensemble is transformed into a scaled model scores (SMS) having values between −1 and +1, wherein,
SMS=(MS−Tₘₐₓ)/(CMS_{K}_{ₘₐₓ}−Tₘₐₓ), . . . when MS>=Tₘₐₓ, and
SMS=(MS−Tₘₐₓ)/(Tₘₐₓ−CMS_{K} min), . . . when MS<Tₘₐₓ,
wherein, Tₘₐₓ, CMS_{K}_{ₘₐₓ}, and CMS_{K}_{ₘᵢₙ} values corresponding to the respective model.

7. The method of claim 6, further comprising calculating an average of all SMS (SMS_{avg}) obtained using all models in the ensemble, wherein
SMS_{avg}=SMS_{avg}*(+1) while using the ensemble of forward models (ENS-MD_{fwd}),
If SMS_{avg}>=0, training biological sample is classified as the second class; and
If SMS_{avg}<0, training biological sample is classified as the first class; and
SMS_{avg}=SMS_{avg}*(−1) while using the ensemble of reverse model (ENS-MDᵣₑᵥ),
If SMS_{avg}>0, training biological sample is classified as the second class; and
If SMS_{avg}<=0, training biological sample is classified as the first class.

8. The method of claim 7, further comprising selecting a final ensemble model (FMₑₙₛ) using the calculated SMS_{avg}, wherein the classification model is one of: the final single model (FM_{single}) or an ensemble of more than one classification models (FMₑₙₛ).

9. The method of claim 3, wherein the first predefined criteria is if a feature (Fⱼ) is observed to have significantly (p<0.1) different median values in the first class compared to the second class in >70% of predefined number of subsets, and if [symbol] >=Q2ₘᵢₙ or [symbol] >=Q2ₘᵢₙ, Fⱼ is added to a set of shortlisted features (SF).

10. The method of claim 1, wherein the relative abundance of each of the microbial taxonomic groups constituting the frugal set of markers, that are common to each of the minimum number of the multiplexed qPCR runs, is determined based on a normalizing factor associated with each multiplexed qPCR run and the relative abundance of associated microbial taxonomic group in the corresponding multiplexed qPCR run.

11. The method of claim 1, wherein determining the relative abundance of each of the microbial taxonomic groups constituting the frugal set of markers in the test biological sample, comprises multiplying a ratio of inferred DNA concentrations of each marker of the frugal set of markers and an inferred DNA concentration of an anchor marker with the median abundance of the anchor marker across the plurality of training biological samples, wherein the anchor marker is selected from the frugal set of markers having a lowest variance in the associated relative abundance.

12. The method of claim 1, wherein each train biological sample and the test biological sample are selected from a group comprising of a vaginal swab sample, a cervical mucus sample, a cervical swab sample, a vaginal swab including swab sample of a vaginal fornix, a urine sample, an amniotic fluid sample, a blood sample, a serum sample, a plasma sample, a placental swab, an umbilical swab, a stool sample, a skin swab, an oral swab, a saliva sample, a periodontal swab, a throat swab, a nasal swab, a vesicle fluid sample, a nasopharyngeal swab, a nares swab, a conjunctival swab, a genital swab, a rectum swab, a tracheal aspirate, and a bronchial swab.

13. The method of claim 1, wherein one or more frugal markers from amongst the frugal set of markers that have a relatively higher median abundance or frequency of occurrence as compared to the median abundances or the frequency of occurrence of each frugal marker in the remaining frugal set of markers, across the plurality of training biological samples are common to the multiplex qPCR runs.
